(19) United States
(12) Patent Application Publication
(10) Pub. No.: US 2008/0194933 A1
(43) Pub. Date: Aug. 14, 2008

(54) Probe For Measuring the Oxygen Content in Biological Tissue, and Catheter With Such a Probe

(75) Inventor: Gerd Kunze, Zwonitz (DE)

Correspondence Address: BROWDY AND NEIMARK, P.L.L.C.;624 NINTH STREET, NW, SUITE 300, WASHINGTON, DC, 20001-5303, US

(73) Assignee: Raumedic AG, Helmbrechts (DE)

(21) Appl. No.: 11/915,509

(22) PCT Filed: May 17, 2006
(86) PCT No.: PCT/EP2006/004664

§ 371 Date: Nov. 26, 2007

(30) Foreign Application Priority Data
Feb. 25, 2005 (DE) 102005024578.1

(51) Int. Cl. A61B 5/1459; A61B 5/1455

(52) U.S. Cl. 600/339; 600/323

(57) ABSTRACT

A probe is used for measuring the oxygen content in biological tissue. The probe comprises at least one optical fibre, which can be proximally optically coupled to a light source on the one hand and to a light sensor on the other. An oxygen-sensitive dye is arranged on and optically coupled to a distal end face of the fibre. A distal fibre portion, including the distal end face, is enclosed together with the dye by an oxygen-permeable, liquid-impermeable membrane which, in the enclosed region, defines a gas compartment surrounding the distal end face with the dye. The probe is a component of a catheter which also comprises a temperature sensor and preferably a pressure sensor. The result is a probe in which the sensitivity of the fibre to disruptive environmental influences at the measuring location is reduced and the scope for interpreting the measurement results is improved.

### BACKGROUND OF THE INVENTION

1. Field of the Invention

The invention relates to a probe for measuring the oxygen content in biological material with at least one optical fibre which can be proximally optically coupled to a light source via one end, and to a light sensor via the other, with an oxygen-sensitive dye which is arranged at a distal end face of the fibre and is optically coupled thereto. In addition, the invention relates to a catheter comprising a probe of this type.

The measurement of oxygen is a subject of great interest, in particular in the field of medicine. Determining in vivo the amount of dissolved oxygen not bound to haemoglobin is important for assessing the supply to the biological material, in particular the tissue. Further examples of biological material to be tested in terms of the oxygen content thereof are body fluids such as blood or liquor. A decisive factor in this process is the oxygen partial pressure in the tested tissue. The partial pressure of the oxygen physically dissolved in the interstitial fluid corresponds to the availability of oxygen on a cellular level. The measurement of oxygen in tissue is used in particular in the cardiovascular and neurosurgical fields, and also in the field of transplant medicine. In the above cases, catheters comprising sensor systems or probes which specifically react to oxygen are predominantly used for measurement.

2. Background Art

A probe of the type mentioned at the outset is known from WO 93/05702 A1. Further probes which measure the oxygen parameters of tissue using fibre optics are known from U.S. Pat. No. 5,673,694, U.S. Pat. No. 5,995,208, U.S. Pat. No. 5,579,774 and the publications cited therein. A further fibre optic oxygen probe is known from J. I. Peterson et al., Anal Chem. 1984, 56, 62-67. Further fibre optic probes are known from U.S. Pat. No. 4,752,115 A, U.S. Pat. No. 5,142,155 A and U.S. Pat. No. 4,861,727 A.

A known measurement method for measuring, using fibre optics, the partial pressure of the physically dissolved. i.e. free oxygen, is dynamic oxygen quenching. In this method, a fluorescent dye embedded in a matrix, for example a platinum complex, is fitted on the distal end of the optical fibre. The fluorescent dye is optically excited via the fibre, generally by laser irradiation which is tuned to the absorption bands of the dye. The dye molecules thus excited change back to the normal state with a time delay, for example in the range between 1 and 60 μs, by emitting light with the same or a red-shifted wavelength. In the presence of oxygen, this transition to the normal state can also take place without radiation by collision processes. In this way, the intensity of the light reflected via the fibre is reduced. This reduction is proportional to the free oxygen in the immediate surroundings of the fluorescent dye. The known fibre optic sensors are extremely sensitive to scattered light and intensity-influencing factors such as hairline cracks or fibre kinking. This sensitivity can be reduced if the phase shift of the light reflected by the fluorescent dye is measured relative to the light radiated in using a lock-in technique. In this method, the fact that long-lived fluorescent states are statistically more susceptible to the radiation-free collision processes of dynamic oxygen quenching is used. The known fibre optic sensors nevertheless exhibit a sensitivity, albeit at a reduced level, to scattered light and intensity-influencing factors even if the lock-in technique is used during measurement. In addition, it has been found that using the known fibre optic sensors in the same tissue region results in very different values for the free oxygen content, which makes interpreting a single measurement therefrom almost impossible.

### SUMMARY OF THE INVENTION

It is therefore an object of the present invention to develop a probe of the type mentioned at the outset in such a way that the sensitivity, of the fibre at the measuring location is reduced with regard to disruptive environmental influences and the scope for interpreting the measurement results is improved.

The object is achieved according to the invention by a probe with a distal fibre portion, including the distal end face together with the dye, is enclosed by an oxygen-permeable, liquid-impermeable membrane which, in the enclosed region, defines a gas compartment enclosing the distal end face with the dye, the dye being provided as a coating on the distal end face and/or on the membrane delimiting the gas compartment or being incorporated into at least a portion of a wall of the membrane.

It has been found according to the invention that forming a gas compartment enclosing the distal fibre portion by an oxygen-permeable and simultaneously liquid-impermeable membrane advantageously increases the measuring volume around the dye. The measuring volume is no longer reduced to the immediate material or tissue surrounding the dye, but extended to the outer surrounding region of the membrane defining the gas compartment. The oxygen partial pressure forming in the gas compartment is thus a measure of the average free oxygen content on the outer surface of the membrane defining the gas compartment. The enlargement of the sensitive volume thus results in a medicinally usable indication of the oxygen supply at a local level, but not at isolated points, in the biological tissue surrounding the probe. Therefore the condition of the tissue can be assessed to a higher standard than by measurement at purely isolated points, which is allowed by the fibre optic measuring methods of the prior art. At the same time, the membrane protects the distal fibre portion in the gas compartment in such a way that the risk of disturbing the measurement at that location is avoided. The robustness of the fibre optic sensor according to the invention is further increased by using the aforementioned lock-in technique. Using the fibre optic sensor, the oxygen content of tissue, but also of other biological material. For example body fluids such as blood or liquor, can be measured. The oxygen-sensitive dye may be, for example, a platinum complex or a ruthenium complex. The oxygen-sensitive dye is either present as a coating or incorporated at least into portions of the membrane wall. The dye must obviously be arranged in such a way that the optical path between the dye molecules and the distal end face of the fibre is as direct as possible. Therefore the dye is preferably directly coated onto the distal end face of the fibre. In contrast to completely filing a volume preceding the distal fibre end face with dye, the arrangement according to the invention of the dye as a coating or component of the membrane wall has the advantage that a light response of the dye is not absorbed by other dye molecules contained in the volume and thus lost in an undesirable manner.

A membrane thickness being uniform where it defines the gas compartment prevents time smearing of the partial pressure measuring signal since the free oxygen molecules take a uniform length of time to diffuse through the membrane. This results in a homogenous sensor characteristic. A uniform membrane thickness does not mean that the membrane thickness is exactly constant over the entire surface of the membrane. Small deviations from an average membrane thickness which do not affect the aforementioned homogeneous sensor characteristic in practice are acceptable. Examples of tolerable deviations of this type are, for example, in the region of 200 μm. An oxygen sensitive dye with long-lasting fluorescence can compensate for disruptive effects caused by deviations in membrane thickness. For this reason, a platinum complex with a fluorescence duration of up to 60 μs is advantageous for a homogeneous sensor characteristic.

A gas compartment being, at least in portions, in the form of a cylinder, the longitudinal axis of which being parallel to or coincides with the fibre axis in the distal fibre portion, can be produced with a membrane that can be manufactured cost-effectively. If the longitudinal axis of the gas compartment is located parallel to the fibre axis and at a distance therefrom, the gas compartment may be formed with a large continuous free volume which is suitable for arranging further components of the probe, in particular sensors. If the axes coincide, this results in a rotationally symmetrical construction which has advantages, in particular in terms of production. When the axes coincide, a configuration is particularly appropriate, in which the distal end face of the fibre with the dye is centred in the gas compartment so there is diffusion length symmetry in terms of the free oxygen which diffuses through the membrane, and this can increase the measuring quality.

A membrane comprising a membrane tube, the ends of which are sealed against penetration of liquid for defining the gas compartment, can be simply produced since the membrane tube can be formed, for example, by cutting a continuous tube to length.

It has been found that materials, from which the membrane is formed, i.e. silicone rubber, PE, PTFE or FEP, are suitable for use in the probe with regard to oxygen permeability and liquid impermeability properties.

The membrane being sufficiently flexible to be deformable under the influence of a gas pressure in the gas compartment adapts well to the Surrounding tissue in such a way that distortion of the measurement is prevented.

The gas compartment being filled with air before insertion of the probe prevents the gas composition from changing during storage of the probe before use. Alternatively, it is possible to fill the probe before use with a gas or a gas mixture, which comprises molecules which are so large that they cannot diffuse through the oxygen-permeable membrane to the exterior. Also in this case, the gas compartment is filled for storage of the probe before use, without changing.

Water vapour permeability of the membrane enables the sensors located in the gas compartment to become adapted more rapidly to the ambient temperature due to the elevated heat capacity of the gas in the gas compartment due to water vapour. In this way, it is possible to reliably measure the temperature within the gas compartment without having to wait for a long time for a thermal equilibrium to be established. Therefore, if it is important to have a high degree of water vapour permeability, the membrane can be formed, in particular, from tetrafluoroethylene-hexafluoropropylene copolymer (FEP). A membrane made of polyethylene (PE) is also water vapour-permeable, albeit to a lower extent than FEP.

A further object is to provide a catheter with which meaningful measurement is achieved by a probe according to the invention.

This object is achieved according to the invention by a catheter with a probe according to the invention, with a temperature sensor for measuring the temperature of the biological material surrounding the catheter, and preferably comprising a pressure sensor for measuring the pressure in the biological material surrounding the catheter.

The temperature sensor allows a thermal dependence of the oxygen content measurement to be compensated. The preferably provided pressure sensor allows an additional pressure measurement to be taken, which, when combined with the oxygen content measurement, provides valuable tissue-specific information. As a result of a combined measurement of this type in which the oxygen content and pressure are measured, the extent to which the dynamics of the oxygen content and the pressure characteristic are correlated can be tested, for example. A correlation of the tissue pressure and the oxygen partial pressure can thus be determined. Detecting different physiological parameters using a single catheter reduces the risk of infection and bleeding in comparison with applying a plurality of individual catheters with separate catheter application points. The preferably partly metallic catheter tip allows it to be seen in image-producing processes, CT for example. As a result, targeted positioning in the desired region is possible. This is required, in particular, to differentiate between a local or global situation in the case of pathophysiological events with reduced oxygen partial pressure values, such as bleeding in the puncture channel, swelling in the region of the catheter location or in the case of local ischaemia. Further advantages of the catheter are those previously mentioned with regard to the probe.

A temperature sensor being arranged, at least in part, in the gas compartment allows good compensation of the temperature dependence of the fibre optic oxygen content measurement, since the temperature is measured at the same location as the oxygen content measurement. The values are also reliable in the case of hypothermia and hyperthermia as a result of the continuous temperature correction.

A catheter tip representing the distal sealing of the membrane tube of the membrane results in a reduction in the number of individual catheter components.

Embodiments of the invention will be described in greater detail in the following with reference to the drawings.

### BRIEF DESCRIPTION OF THE DRAWINGS

FIG. 1 a schematic longitudinal cross-section of a probe for measuring the oxygen content in biological tissue;

FIG. 2 a view similar to FIG. 1 of the probe in which a distal fibre portion of an optical fibre is pushed further into a gas compartment defined by a membrane:

FIG. 3 a cross-section along line III-III in FIG. 2;

FIG. 4 a cross-section similar to that of FIG. 3 through a further embodiment of a probe.

FIG. 5 a longitudinal section through a catheter with a further embodiment of a probe for measuring the oxygen content in biological tissue;

FIG. 6 a schematic cross-section along line VI-VI in FIG. 5;

FIG. 7 a cross-section similar to that of FIG. 5 through a further embodiment of a catheter, and

FIG. 8 a front view according to the arrow VIII in FIG. 7.

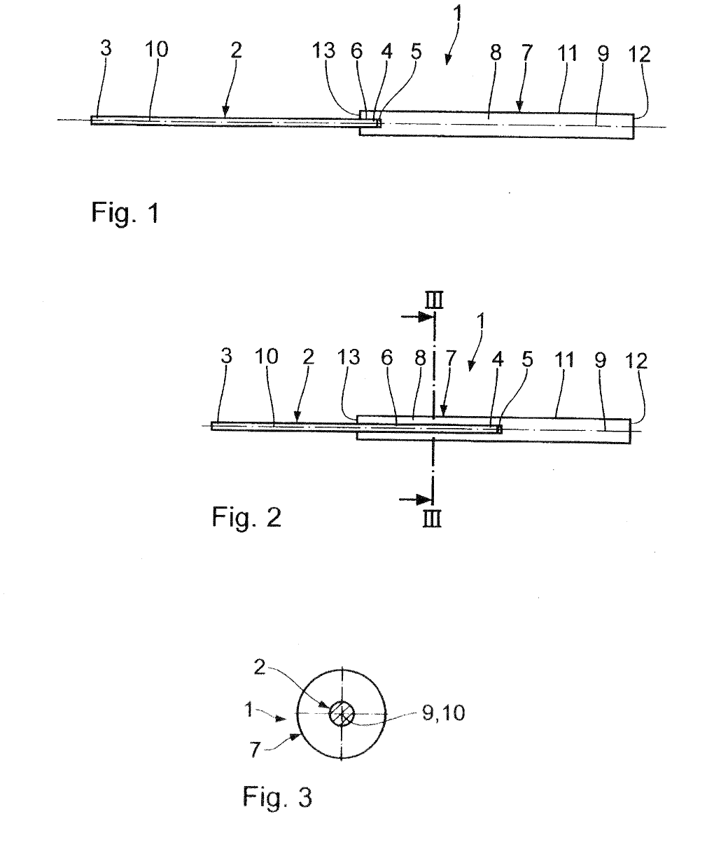

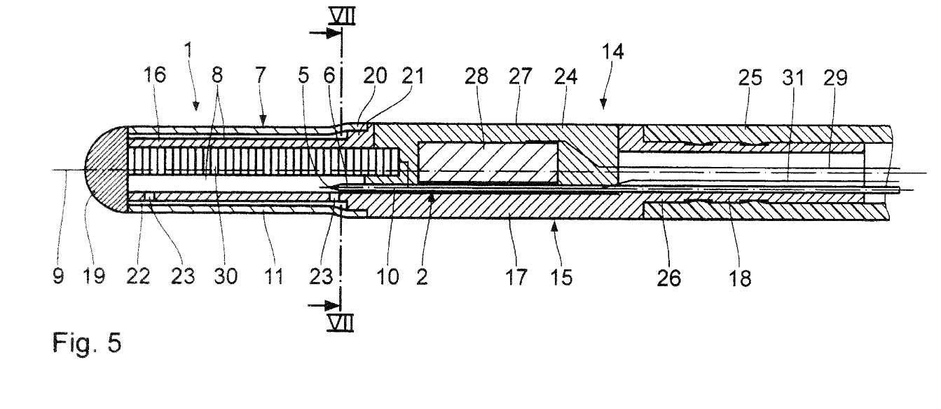

FIGS. 1 to 3 show a first embodiment of a probe for measuring the oxygen content in biological tissue. The probe 1 can be a component of a catheter, for example of the type shown in FIG. 5.

### DESCRIPTION OF THE PREFERRED EMBODIMENTS

The probe 1 comprises an optical fibre 2. A proximal end 3, which is remote from the biological tissue to be tested can be optically coupled to a light source on the one hand, and a light sensor on the other. The optical fibre 2 may be a single fibre or a fibre bundle.

An oxygen-sensitive dye 5 is arranged on a distal end face 4 of the optical fibre 2 which faces the biological tissue to be tested. The dye 5 is optically coupled to the distal end face 4 of the optical fibre 2. The distal end face 4 is coated with the dye 5. A distal fibre portion 6 including the distal end face 4, together with the dye 5, is enclosed by an oxygen-permeable, liquid-impermeable membrane 7. The membrane 7 is configured to be water vapour-permeable. The membrane defines, in the enclosed region, a gas compartment 8 which surrounds the distal end face 4 with the dye 5. As an alternative to coating the distal end face 4 with the dye 5, it is possible to coat the inner wall of the membrane 7 with the dye 5 at least in some regions. The selected regions to be coated are those which can be “seen” by the distal end face 4, i.e. to which there is a direct optical path from the distal end face 4. In a further variant it is possible to incorporate the dye 5 into the wall of the membrane 7.

The membrane 7 has a uniform thickness where it defines the gas compartment 8. The permissible deviation in membrane thickness from a pre-determined value is a function of the desired measuring dynamics of the oxygen partial pressure. Deviations of, for example, 200 μm have been found to be tolerable for measurements carried out in brain tissue. In the probe shown in FIGS. 1 to 3, the gas compartment 8 is in the form of a cylinder. A gas compartment longitudinal axis 9 coincides with a fibre axis 10 at least in the distal fibre portion 6.

In the embodiment shown in FIGS. 1 to 3, the membrane 7 is made of silicone rubber. Alternatively, the membrane 7 may also be made of one of the following materials: polyethylene (PE), Teflon (PTFE) or tetrafluoroethylene-hexafluoropropylene copolymer (FEP). The membrane 7 is sufficiently flexible to be deformable under the influence of gas pressure in the gas compartment 8. The shape of the gas compartment 2 can thus be adapted to the respective gas pressure present as a function of ambient pressure.

According to the application, the fibre 2 can be positioned in different ways relative to the membrane 7 in the probe 1. In the position in FIG. 1, the fibre 2 is inserted merely a short way into the gas compartment 8 in such a way that the distal fibre portion 6 enclosed by the membrane 7 is short compared to the length of the gas compartment 8. In the position shown in FIG. 2, the fibre 2 is inserted further into the gas compartment 8 in such a way that the distal fibre portion 6 inserted therein is approximately half as long as the gas compartment 8. In the position shown in FIG. 2, the distal end face 4 with the fibre 5 is located symmetrically centrally in the gas compartment 8 so there is diffusion length symmetry in terms of the free oxygen diffusing through the membrane 7.

The probe 1 is used in the following manner:

The probe 1, optionally with a catheter comprising said probe, is initially brought into the measuring position in vivo in a patient. The gas compartment 8 is filled with air before the probe 1 is used. Both the light source and the light sensor are proximally coupled to the fibre 2. The membrane 7 is surrounded externally by the biological tissue of the patient. Free oxygen, i.e. oxygen not bound to haemoglobin, can diffuse through the membrane 7 from the outside, thus penetrating the gas compartment 8. Since the gas compartment 8 is closed off from the outside in a liquid-tight manner, neither liquid nor tissue can penetrate the gas compartment 8.

The dye 5 is tuned to the wavelength of the coupled light in such a way that, as a result of the light coupled into the dye 5 under the influence of the oxygen molecules present in the gas compartment 8, light, in an amount thereof which can be measured by the light sensor, fed back from the dye 5 into the optical fibre 2 is a function of the concentration of the free oxygen in the gas compartment 8. The uniform thickness of the membrane 7 defining the gas compartment 8 correspondingly ensures a uniform penetration time of the free oxygen from the biological tissue surrounding the membrane 7 into the gas compartment 8. Measuring errors due to different penetration times thus cannot arise.

The amount of light fed back from the dye 5 into the fibre 2 as a result of the light coupled from the light source into the fibre 2 is measured using the light sensor. This amount of fed-back light is a measure of the oxygen content in the gas compartment 8 and is thus a measure of the oxygen not bound to haemoglobin, i.e. free oxygen in the biological tissue surrounding a the membrane 7. Alternatively, it is possible to measure the phase shift of the fed-back light as a function of the phase of the coupled light, using the lock-in technique for example. Since long-lived states of the dye 5 are statistically more susceptible to an oxygen-induced radiation-free transition to the normal state by a collision process, the average duration of the fluorescent states, which contribute to the fed-back light, is shifted, which in turn results in a measurable phase shift relative to the radiated signal which can be used as a lock-in reference.

In the configuration shown in FIGS. 1 to 3, the membrane 7 is configured as one piece. The material of the membrane 7 provides a seal against the optical fibre 2 in the region of the fibre entry into the gas compartment 8.

In a variant of the probe 1 which, for the sake of simplicity, will also be described with reference to FIGS. 1 to 3, the membrane 7 comprises a membrane tube 11 which defines the jacket wall of the cylindrical gas compartment 8. An end-face end of the membrane tube 11 which is remote from the fibre has a sealing cover 12. The sealing cover can be made of the same material as the membrane tube 11. Alternatively, it is possible to produce the sealing cover 12 from a different, in particular completely fluid-impermeable, material to that of the membrane tube 1 as it is sufficient for the membrane tube 11 to be oxygen-permeable. The membrane tube 11, on the side facing the fibre, is sealed against the fibre 2 by a sealing ring 13 which can be made of the same material as the sealing cover 12.

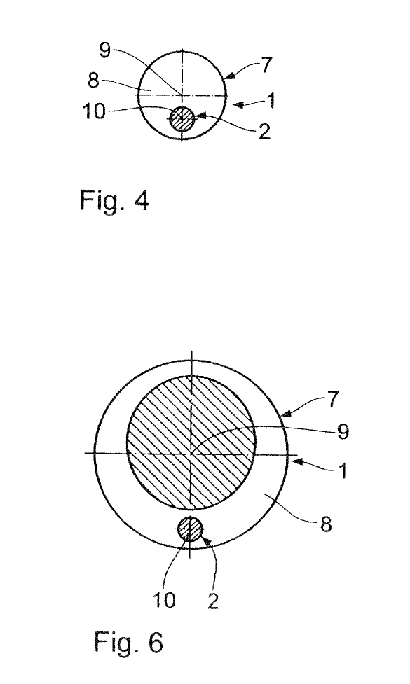

The configuration of the probe 1 in FIG. 4 differs from that of FIGS. 1 to 3 merely in that the gas compartment longitudinal axis 9 of the probe in FIG. 4 does not coincide with the fibre axis 10 in the gas compartment 8, but is parallel thereto. In this way, the gas compartment 8 in the configuration shown in FIG. 4 has a greater continuous free volume in which further components, for example further sensors, may be accommodated.

FIGS. 5 and 6 show a catheter 14 with a further configuration of a probe 1. The catheter 14 is described in the following only if it differs to what was previously stated in relation to FIGS. 1 to 4. Components which correspond to those previously described with reference to FIGS. 1 to 4 have the same reference numerals and are only described if they differ in terms of construction and function from the components in FIGS. 1 to 4. The catheter 14 has a housing 15. In the configuration shown said housing is made from titanium, but it may also be made of another material. The housing 15 is made of one piece and is structurally divided into a distal housing portion 16, a centre housing portion 17 and a proximal housing portion 18. The distal housing portion 16 is covered at its distal end by an atraumatic catheter tip 19. At the periphery of the distal housing portion 16, the catheter tip 19 merges into the membrane tube 11 of the membrane 7.

The catheter tip 19 is a sealing cover of the membrane 7. A proximal peripheral end portion 20 of the membrane 7 is pushed onto a peripheral step 21 of the centre housing portion 17. The outer diameter of the peripheral step 21 is slightly greater than the inner diameter of the membrane tube 11.

Between the membrane tube 11 and the distal housing portion 16, there is an annular space 22 which is part of the gas compartment 8 and is connected by perforations 23 to a cylindrical interior of the distal housing portion 16 which is also part of the gas compartment 8. The distal fibre portion 6 of the optical fibre 2 with the dye 5 is inserted into said interior. Further on, the fibre 2 initially passes through a sealing body 24 which is inserted in the centre housing portion 17 and can be made of, for example, silicone rubber. Further on, the fibre 2 passes through a cylindrical interior of the proximal housing portion 18 and also a catheter tube 25. The catheter tube is pushed onto a peripheral step 26 formed in the proximal housing portion 18.

An outer wall 27 of the sealing body 24 is arranged in a housing window in the centre housing portion 17 and is aligned with the surrounding outer wall of the centre housing portion 17. A pressure sensor 28 is arranged in the sealing body 24. The pressure sensor 28 is connected to a central control and evaluation unit (not shown) by a signal line 29 which extends through the sealing body 24, the proximal housing portion 18 and the catheter tube 25.

As in the configuration shown in FIG. 4, in the probe 1 shown in FIGS. 5 and 6, the gas compartment longitudinal axis 9 does not coincide with the fibre axis 10 so there is a large continuous free volume in the interior defined by the distal housing portion. A temperature sensor 30 is arranged in said interior. A proximal end of the temperature sensor 30 is inserted into the sealing body 24 in a sealed manner. A signal line 31 connects the temperature sensor 30 to the central control and evaluation unit. The signal line 31 also passes through the sealing body 24, the proximal housing portion 18 and the catheter tube 25.

The function of the catheter 14 will be described in the following only if there is a difference to the use of the probe 1 of FIGS. 1 to 4. After the catheter 14 is brought into the measuring position in the patient, the oxygen content of the biological tissue surrounding the catheter 14 is measured with the probe 1 according to the above description with regard to the configuration shown in FIGS. 1 to 4. At the same time, the pressure exerted by the biological tissue on the pressure sensor 28 via the outer wall 27 is measured by the pressure sensor 28, and the temperature in the gas compartment 8 is measured by the temperature sensor 30. The measurement values are fed, via the signal lines 29 and 31, to the central control and evaluation unit, to which the light source and the light sensor of the probe 1 are connected. After thermal equilibrium has been established, the temperature in the gas compartment 8, which is measured by the temperature sensor 30, corresponds to the temperature of the biological tissue surrounding the distal housing portion 16 of the catheter 14. Water vapour which permeates through the membrane tube 11 and penetrates the gas compartment 8 is responsible for this temperature equalisation, and is also the basis for the rapid temperature measurement.

As a result of the temperature measured by the temperature sensor 30, the temperature dependence of the water vapour partial pressure in the oxygen partial pressure measurement can be taken into account via the optical fibre 2.

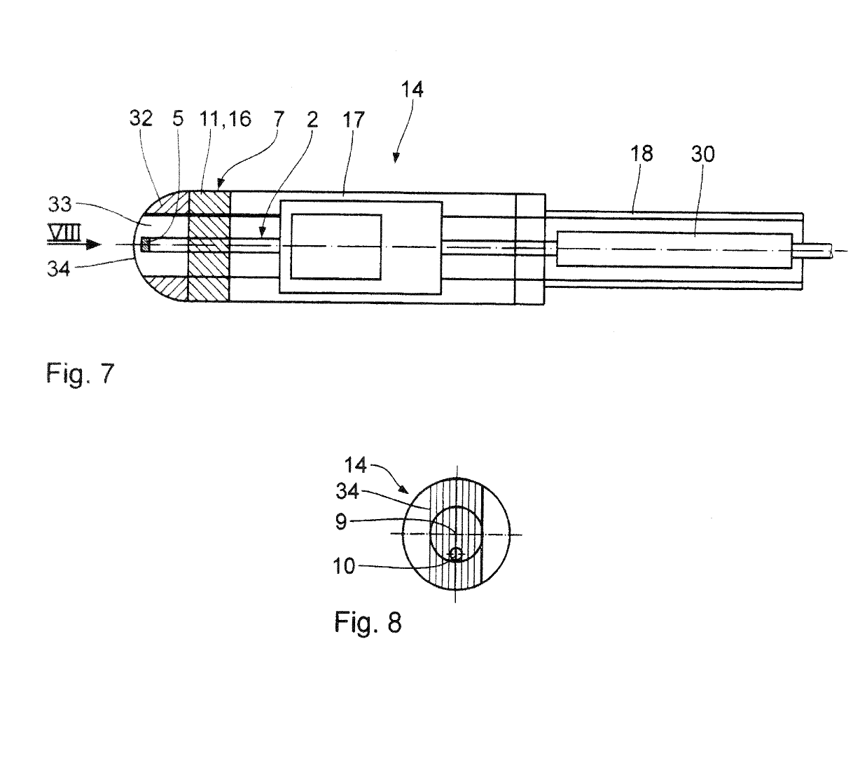

FIGS. 7 and 8 show a further configuration of a catheter comprising a probe for measuring the oxygen content in biological tissue. Components which have previously been described with reference to FIGS. 1 to 6 have the same reference numerals and will not be explained again individually.

The catheter 14 shown in FIGS. 7 and 8 differs from that of FIGS. 5 and 6 pre-dominantly in the shape of the membrane 7 and the arrangement of the sensors. In the configuration shown in FIGS. 7 and 8, a catheter tip 32 is not made of solid material as in the configuration shown in FIGS. 5 and 6, but has an inner recess 33 which is part of the gas compartment 8. The recess 33 is distally covered by an end face membrane portion 34 which is made of the same material and has the same material thickness as the membrane tube 11. The membrane portion 34 merges seamlessly at its edge into portions of the catheter tip 32 surrounding said membrane portion in such a way that the membrane portion 34, together with the portions surrounding said membrane portion, forms the atraumatic catheter tip. The membrane portion 34 is indicated in the front view in FIG. 8 by parallel shading. The optical fibre 2 with the dye 5 is inserted into the recess 33 of the catheter tip 32 in the configuration shown in FIGS. 7 and 8.

In the configuration shown in FIGS. 7 and 8, the gas compartment longitudinal axis 9 also does not coincide with the fibre axis 10, but is arranged at a distance therefrom and parallel thereto.

In the configuration in FIGS. 7 and 8, the temperature sensor 30 is not arranged in the gas compartment 87 but in the proximal housing portion 18.

The function of the catheter 14 shown in FIGS. 7 and 8 corresponds to that of the catheter shown in FIGS. 5 and 6. In the case of the catheter 14 shown in FIGS. 7 and 8, the temperature is measured in the region of the proximal housing portion 18 in such a way that, to correctly allow for the temperature dependence of the water vapour partial pressure, it is necessary for the temperature of the biological tissue in the region of the proximal housing portion 18 to correspond to the temperature in the region of the distal housing portion 16.

Platinum or ruthenium complexes may be used as the dye 5. Typical fluorescence durations of platinum complexes are 60 μs at 0% air saturation and 20 μs at 100% air saturation. Typical fluorescence durations of ruthenium are approximately 6 μs at 0% air saturation and approximately 4 us at 100% air saturation.

1. A probe (1) for measuring the oxygen content in biological material
with at least one optical fibre (2) which can be proximally optically coupled to a light source via one end, and to a light sensor via the other,
with an oxygen-sensitive dye (5) which is arranged at a distal end face (4) of the fibre (2) and is optically coupled thereto,
wherein a distal fibre portion (6), including the distal end face (4) together with the dye (5), is enclosed by an oxygen-permeable, liquid-impermeable membrane (7) which, in the enclosed region, defines a gas compartment (8) enclosing the distal end face (4) with the dye (5), the dye (5)
being provided as a coating at least on one of the group comprising the distal end face (4) and/ the membrane (7) delimiting the gas compartment (8).

2. A probe according to claim 1, wherein the thickness of the membrane (7) is uniform where it defines the gas compartment (8).

3. A probe according to claim 1, wherein the gas compartment (8) is, at least in portions, in the form of a cylinder, the longitudinal axis (9) of which is parallel to or coincides with the fibre axis (10) in the distal fibre portion (6).

4. A probe according to claim 1 wherein the membrane (7) comprises a membrane tube (11), the ends (12, 13) of which are sealed against penetration of liquid for defining the gas compartment (8).

5. A probe according to claim 1, wherein one material of the group of
silicone rubber,
PE,
PTFE,
FEP.
forms the membrane (7).

6. A probe according to claim 1, wherein the membrane (7) is sufficiently flexible to be deformable under the influence of a gas pressure in the gas compartment (8).

7. A probe according to claim 1, wherein the gas compartment (8) is filled with air before insertion of the probe (1).

8. A probe according to claim 1, wherein the membrane (7) is configured to be water vapour-permeable.

9. A catheter (14)
with a probe (1) according to claim 1,
with a temperature sensor (30) for measuring the temperature of the biological material surrounding the catheter.

10. A catheter according to claim 9, wherein the temperature sensor (30) is arranged, at least in part, in the gas compartment (8).

11. A catheter according to claim wherein the membrane (7) comprises a membrane tube (11), the ends (12, 13) of which are sealed against penetration of liquid for defining the gas compartment (8), and a catheter tip (19; 32) represents the distal sealing of the membrane tube (11) of the membrane (7).

12. A catheter (14) according to claim 9, comprising a pressure sensor (28) for measuring the pressure in the biological material surrounding the catheter (14).

13. A probe (1) for measuring the oxygen content in biological material
with at least one optical fibre (2) which can be proximally optically coupled to a light source via one end, and to a light sensor via the other,
with an oxygen-sensitive dye (5) which is arranged at a distal end face (4) of the fibre (2) and is optically coupled thereto,
wherein a distal fibre portion (6), including the distal end face (4) together with the dye (5), is enclosed by an oxygen-permeable, liquid-impermeable membrane (7) which, in the enclosed region, defines a gas compartment (8) enclosing the distal end face (4) with the dye (5), the dye (5) being incorporated into at least a portion of a wall of the membrane (7).
